(19) United States
(12) Patent Application Publication
(10) Pub. No.: US 2025/0041090 A1
(43) Pub. Date: Feb. 6, 2025

(54) STENT DEVICE

(71) Applicant: Vascutek Limited, Renfrewshire (GB)

(72) Inventors: Gary McDonald, Lanarkshire (GB); Vincent Nelis, Renfrewshire (GB)

(21) Appl. No.: 18/926,860

(22) Filed: Oct. 25, 2024

Related U.S. Application Data
(63) Continuation of application No. 17/311,897, filed on Jun. 8, 2021, now Pat. No. 12,127,961.

(30) Foreign Application Priority Data
Dec. 20, 2018 (GB) 1820898.3

(51) Int. Cl. A61F 2/97; A61F 2/07; A61F 2/89; A61F 2/95; A61F 2/966

(52) U.S. Cl. CPC A61F 2/97; A61F 2/89; A61F 2/966; A61F 2/07; A61F 2002/9511; A61F 2220/0075; A61F 2230/0095; A61F 2250/0039

(57) ABSTRACT

The present invention relates to a stent device comprising a sleeve formed with a plurality of compactable spring ring elements arranged along its length, the sleeve having compacted and expanded states. Each said ring element has an undulating profile at the surface of the sleeve so that adjacent ring elements at least partially overlap along the longitudinal extent of the device, the ring elements being compactable against their natural resilience to reduce the outer diameter of the sleeve for allowing housing of the compacted stent device in a frangible sheath, with adjacent ring elements being inter-coupled so as to substantially maintain their axial spacing between the compacted and expanded states.

### CROSS REFERENCE TO RELATED APPLICATIONS

This application is a Continuation of and claims the benefit of priority under 35 USC 120 to U.S. patent application Ser. No. 17/311,897, filed on Jun. 8, 2021, which claims the benefit of priority under 35 USC 371 to International App. No. PCT/GB2019/053239, filed on Nov. 15, 2019, which claims the benefit of Great Britan App. No. GB 1820898.3, filed Dec. 20, 2018, the entirety of each is hereby incorporated by reference.

The present invention relates to a stent device.

In this connection, current treatment methods for aortic dissections and aneurysms predominantly utilize conventional surgical grafts and open surgery. Although endovascular treatment methods are also possible, the complexity of maintaining profusion to all major branch vessels radiating from the top of the aortic arch by purely an endovascular approach, means that endovascular treatment methods are currently very limited.

Furthermore, the use of conventional surgical grafts often necessitates a full thoracotomy, namely a major surgical opening of the chest cavity, which typically necessitates a Coronary Artery Bypass and the need to induce hypothermia and cardiac arrest. Undertaking such surgical procedures is not without risk of further complications.

Moreover, known endovascular stent devices and their delivery systems rely on the use of an internal delivery support shaft, typically having an integral moulded tip at its end to facilitate insertion, as well as mounting loops and release wire to support and deploy the stent device. This type of arrangement allows the stent device to be suspended from the delivery system at the tip end. It is held there until unsheathing has been performed, after which it can then be released from the delivery system.

After release, which is typically by the removal of a release wire, the supporting internal central shaft and tip assembly must then be fully removed from within the stent device by retracting these items through the inside of the device lumen. These items must be removed carefully to overcome the potential risk of dislodging the previously deployed device by unintentional snagging. To provide this functionality, the delivery system typically necessitates other adjunctive elements such as a guide-wire, which would pass through the internal lumen of the shaft and tip moulding.

Such components prohibit anastomosing the non-stented end of the device to either an adjunctive device or native vessel prior to deployment of the stented section, should that be required.

An object of the present invention is to provide an improved stent device that can alleviate problems associated with what is currently available.

According to the present invention there is provided a stent device comprising:—a sleeve formed with a plurality of compactable spring ring elements arranged along its length, the sleeve having compacted and expanded states; wherein each said ring element has an undulating profile at the surface of the sleeve so that adjacent ring elements at least partially overlap along the longitudinal extent of the device, the ring elements being compactable against their natural resilience to reduce the outer diameter of the sleeve for allowing housing of the compacted stent device in a frangible sheath, with adjacent ring elements being inter-coupled so as to substantially maintain their axial spacing between the compacted and expanded states.

In this way, the stent device can be compacted so as to adopt a reduced diameter for insertion purposes, the sleeve being in a compressed constrained state when provided within such a frangible sheath. On release from the sheath, the sleeve will, by virtue of the ring elements expand to adopt a larger diameter.

Providing the stent device in a compactable form affords it an integral column stiffness so that internal delivery mechanisms for deploying the stent device can be dispensed with. This simplifies the stent deployment process and importantly bypasses the risks associated with procedures which involve withdrawing such internal delivery mechanisms, in particular dislodgement of the stent device that has just been inserted. The externalized nature of the stent device deployment that is made possible by the stent device of the present invention moreover enhances the ability for anastomosing the stent device to other adjunctive devices or native vessels.

Preferably, the undulating profile of each ring element extends around the surface of the sleeve at the sleeve's periphery. Whilst different undulating ring element profiles may be employed, such as “Z” shapes, each ring element preferably has a hyperbolic paraboloid profile, whereby it is substantially saddle-shaped. In this way, in the compacted configuration, the ring elements can be imbricated, so that they stack axially along the length of the sleeve in an overlapping configuration. The overlapping nature of the ring elements in the compacted configuration enhances the provision of a column stiffness facilitating use with deployment apparatus. In this regard, when in a compacted configuration, the peaks of one ring element overlie the valleys of an adjacent ring element. As such, the close abutment of portions of adjacent ring elements in the compacted configuration affords a column stiffness to the device.

The ring elements are preferably arranged such that adjacent ring elements are mounted along the sleeve for maintaining relative ring position. In this way, ring position is maintained across the compaction and deployment states of the stent device.

Conveniently, the ring elements are inter-coupled by way of mounting to the sleeve material. Preferably, where adjacent ring elements overlap axially, their circumferential spacing, when in an open configuration of the device, is less than or equal to the maximum change in axial extent of each ring element when moving from an expanded to a compacted configuration. As such, when in the compacted state, fabric between adjacent ring elements is in tension, preventing adjacent rings impinging axially on one another.

Conveniently, the sleeve material is a fabric such as for example gel coated polyester.

Preferably, the ring elements are formed of a nitinol wire. Conveniently, the wire has a diameter in the range 0.08 to 0.24 mm.

The stent device furthermore may have a soft tip at a proximal end. In this respect the soft tip may extend beyond the end of the sheath, when the device is housed in a sheath. The soft tip enhances the functionality of the stent device, affording it atraumatic characteristics allowing it to be deployed without an internal delivery shaft as with known arrangements.

As such, a portion of the proximal end of the stent device is exposed and may be covered in one or more of soft suture or PTFE thread to form an atraumatic tip. In this regard, the soft tip may be formed from stent material at the end of the device. It may comprise a heavy suture on a saddle profile. Further it may comprise one or more additional layers of suture over one or more of the ring elements at the proximal end of the stent device.

According to a further aspect of the present invention there is provided a stent device comprising a sleeve formed with a plurality of compactable spring ring elements arranged along its length, wherein a soft end tip is formed at a proximal end of the sleeve, the soft tip end comprising a portion of the sleeve covered in one or more of soft suture material or PTFE thread.

In this regard, the soft tip may be formed from stent material at the end of the device, folded into a ring and held with suture.

The soft tip may comprise a heavy suture on a ring element having a saddle profile.

Further, it may comprise one or more additional layers of suture over one or more of the ring elements at the proximal end of the sleeve. With the ring elements having a saddle or hyperbolic paraboloid profile, the soft tip is naturally rounded to optimise its atraumatic characteristics.

Embodiments of the present invention will now be describe by way of example and with reference to the following drawings, of which: —

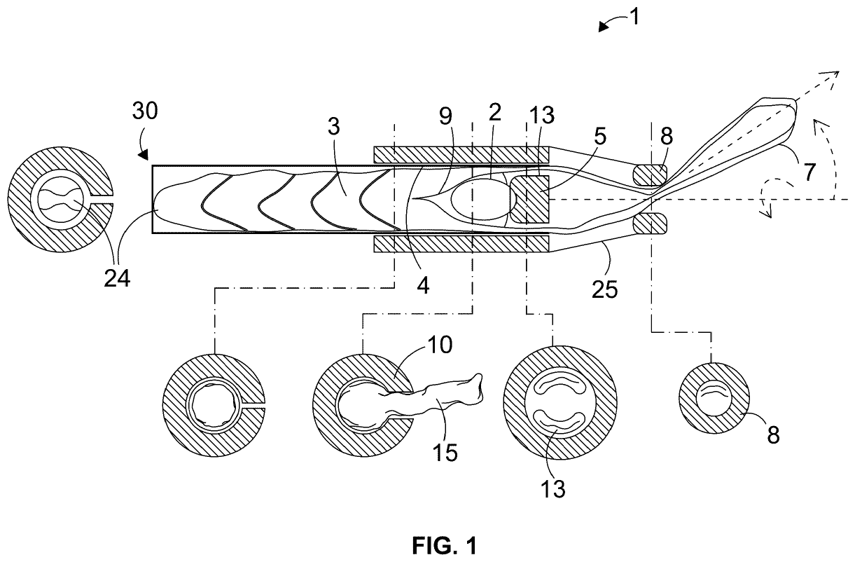

FIG. 1 shows a cross-sectional view of compatible deployment apparatus into which a sheathed stent device of the present invention is located;

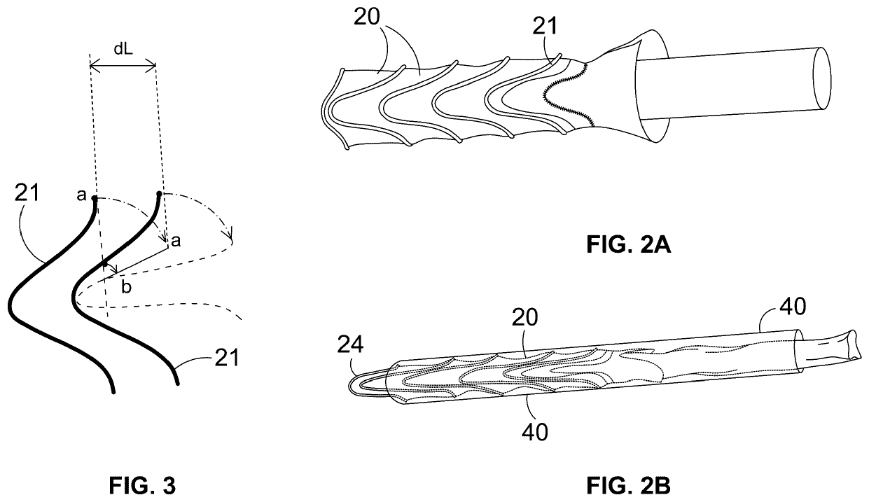

FIGS. 2a and 2b show views of a stent device of the present invention; and

FIG. 3 is a schematic view showing adjacent ring elements of a stent device of the present invention.

In this connection, FIG. 1 shows a cross-sectional view of deployment apparatus 1, compatible with a stent device of the present invention, the deployment apparatus having a body 2 into which a sheathed stent device 3 of the present invention is located.

In this regard, the body has a bore 4, dimensioned to allow the sheathed stent device 3 to sit within the bore, but not so tight so as to prevent the sheath material from moving relative to the bore and the stent device.

As regards the stent device, as shown unsheathed and sheathed respectively in FIGS. 2a and 2b, this preferably comprises a lumen or sleeve 20 of fabric, typically gel coated polyester, fitted with a series of spring like “stent elements”, typically having ring elements 21 formed from nitinol wire in the shape of an undulating “Z” shaped stent or in the preferred embodiments a saddle ring, namely a hyperbolic paraboloid.

Multiple such ring elements 21 are located along the axis of the lumen and these are attached circumferentially to the fabric by sutured thread, to form the stented device section, which has the capacity to be constrained into a significantly smaller diameter tube, namely sheath 40.

When compacted into the small calibre sheath 40 as shown in FIG. 2b, the sheathed stent device (with an appropriate selected oversize) can be readily inserted into the lumen of a branch vessel. Removing the sheath from the stent device enables it to be deployed into the native vessel, where the stented section expands radially outwards.

The radially expanding stent elements contact and push onto the internal vessel wall to create a snug fitting non-sutured sealed junction.

The overlapping nature of the ring elements in the compacted configuration affords the sleeve with a column stiffness facilitating use with compatible deployment apparatus, such as shown in FIG. 1.

More specifically, providing the stent device in a radially compactable form affords it an integral column stiffness so that internal delivery mechanisms for deploying the stent device can be dispensed with. This simplifies the stent deployment process and importantly bypasses the risks associated with procedures which involve withdrawing such internal delivery mechanisms, in particular dislodgement of the stent device that has just been inserted.

In this connection, the ring elements are preferably arranged within the sleeve such that the axial spacing of adjacent elements is maintained. In this way, ring element position is maintained across the compaction and deployment states of the stent device.

As shown in FIG. 3, the ring elements 21 are connected to the sleeve material such that where adjacent ring elements overlap axially, their circumferential spacing a-b, when in an open configuration of the device, is less than or equal to the maximum change in axial extent dL of each ring element when moving from an expanded to a compacted configuration. As such, when in the compacted state, fabric between adjacent ring elements is in tension, preventing adjacent rings impinging axially on one another.

In this connection, the device may be configured with a relatively high saddle height, namely a relatively large axial difference between the peaks and the troughs of the ring. Further, the ring inter spacing is preferably less than the saddle height, to provide an overlap of the peaks and valleys of adjacent rings. This property in combination with an adjacent section of supported fabric is utilised to maintain the position of the stent device relative to the body 2, prior to and during the unsheathing process.

As shown in FIG. 1, at or near the distal end of the stented section, there may be provided a section of flexible crimped fabric 15, typically gel coated polyester, which is joined and attached by suturing to form a blood tight continuous endoprothesis lumen. In some embodiments this section may also include a “Y” branch lumen. The non-stented section is provided to enable an endoprothesis to be joined by suturing to either the main prosthesis body or alternatively to a healthy section of native vessel, to reinstate blood profusion to the native branch vessel.

The sheath is preferably thin walled (typically a PTFE material), which has an inherent preposition to tear linearly, without the need for additional grooves or perforations. The sheath may have three sections: a proximal circular section, which has a length slightly longer than the length of the compacted stented section, a tail section at its distal end and a mid-section, where the circular section splits and propagates into the two tail elements.

With the compatible deployment apparatus shown in FIG. 1, these flat ribbon like tail elements 7 originate from the end of the circular section and can be formed by folding. The formed tails are fed through or past a restriction 5 within the body 2 of the compatible deployment apparatus and out into a separate strap element, where they can be tied together to form a singular user interface for sheath removal.

As shown in FIG. 1, the restriction 5 in the bore 2 is configured to obstruct travel of the stent device. The restriction however allows stent device sheath material, namely the tails 7 to pass the restriction for access at the distal end of the body 2.

Whilst any suitable means may be employed to allow passage of the sheath material past the restriction, the compatible deployment apparatus has two arcuate apertures 13 in the face of the restriction 5, the apertures extending longitudinally in the axial direction of the body. The apertures are substantially circumferential and subtend an angle of 90 to 120 degrees. In this connection, the apertures each provide passage for a tail of sheath material 7, the sheath material being split within the bore 4 at point 9.

The body has a side window 10 for allowing the sheathed stent device to be positioned within the body with a crimped section 15 of the stent device exiting the body to the side via the window. The side window hence provides a pathway for the non-stented device fabric 15 to pass through out from the confines of body 2 substantially perpendicularly to the axis of the sheathed sheath within the bore, enabling access to the distal end of the stent device. This end can hence be trimmed in length to suit individual patient anatomy and facilitates suturing to an adjunctive graft or native vessel.

Once the stent device has been sufficiently deployed, it can be removed from the body 2 of the compatible apparatus.

With the above compatible deployment apparatus, the body 2 holds and supports the sheathed stent device 3 to enable the proximal compacted section to be inserted into either a native vessel or an adjunctive stent device body, so that it can be held for subsequent unsheathing and deployment to then enable vessel profusion to be reinstated.

This simplifies the delivery system in terms of its complexity, which together with the reduction in components provides the user with fewer procedural steps and potential risks, enabling a more time efficient and simplified device deployment.

The internal arrangement within the body enables controlled parting of the sheath when the user pulls the strap element. When the sheath is pulled across the internal bore restriction, the circular lumen aspect of the sheath is caused to continue to split, propagating along the two tail elements 7. Simultaneously, the movement applied at the strap is transmitted to the proximal end of the sheath, causing it to slide over the stent device, enabling the compacted stent device to be relieved from its radial constraint. In doing so, the stent device opens and engages the internal lumen of the vessel.

As shown in FIGS. 1, 2a and 2b, the stent may have an integral stent device tip 24 feature. This may be provided at the proximal end of the stented region of the stent device 3, which when compacted within the sheath constraint can protrude beyond the end of the sheath to partially expose said compacted stent device elements covered in soft suture (or PTFE thread) to provide an atraumatic tip like feature.

What is claimed is:

1. A stent device comprising:
a sleeve having a plurality of compactable spring ring elements disposed along its length, the sleeve having compacted and expanded states;
wherein each spring ring element has an undulating profile with adjacent ring elements at least partially overlapping along the length of the device,
the spring ring elements being compactable against their natural resilience to reduce an outer diameter of the sleeve for housing of the compacted stent device in a frangible sheath;
wherein the spring ring elements are inter-coupled such that where adjacent ring elements overlap axially, a circumferential spacing of the adjacent ring elements, when in an open configuration of the device, is less than or equal to a maximum change in axial extent of each ring element when moving from an expanded to a compacted configuration; and
when in the compacted state, the fabric between adjacent spring ring elements is in tension.

2. The stent device as claimed in claim 1, wherein adjacent spring ring elements maintain their axial spacing from the proximal end to the distal end between the compacted and expanded states.

3. The stent device as claimed in claim 1, wherein the undulating profile of each spring ring element extends circumferentially around the surface of the sleeve.

4. The stent device as claimed in claim 1, wherein when in a compacted configuration, a plurality of peaks of one spring ring element overlie a plurality of valleys of an axially adjacent spring ring element.

5. The stent device as claimed in claim 1, wherein the spring ring elements are formed of a nitinol wire with a diameter in the range 0.08 to 0.24 mm.

6. The stent device as claimed in claim 1, wherein the spring ring elements have an arcuate profile.

7. The stent device as claimed in claim 1, wherein the spring ring elements have a hyperbolic paraboloid profile.

8. The stent device as claimed in claim 1, wherein the spring ring elements have a saddle profile.

9. The stent device as claimed in claim 8, wherein a distance between a peak and a trough of a first spring ring element defines a saddle height, with the distance axial distance between adjacent ring elements being less than the saddle height.

10. The stent device as claimed in claim 1, further comprising a non-stented section at a distal end.

11. The stent device as claimed in claim 10, wherein the non-stented section includes a tail element of folded fabric.

12. The stent device as claimed in claim 11, further including a deployment apparatus configured to receive the stent device therein, the deployment apparatus having a body with a window providing a pathway for a crimped portion of the stent device to pass throughout.

13. The stent device as claimed in claim 12, wherein the deployment apparatus is configured to receive the stent device within the body, with the crimped portion of the stent device passing substantially perpendicularly to the axis of the stent device within the body.

14. The stent device as claimed in claim 12, wherein the deployment apparatus includes a restriction configured to obstruct travel of a stent device within the body, but allow passage of the tail element out of a distal end of the body.

15. The stent device as claimed in claim 14, wherein the sheath fabric is configured to be split within the deployment apparatus.
